(19) United States
(12) Patent Application Publication
(10) Pub. No.: US 2024/0265891 A1
(43) Pub. Date: Aug. 8, 2024

(54) ANALYSIS AND AUGMENTATION OF DISPLAY DATA

(71) Applicant: Brainlab AG, Munich (DE)

(72) Inventors: Nikolaus NEUMAIER, Munich (DE); Nils Frielinghaus, Munich (DE); Christoffer Hamilton, Munich (DE)

(21) Appl. No.: 18/559,744

(22) PCT Filed: Jun. 11, 2021
(86) PCT No.: PCT/EP2021/065821

§ 371 Date: Nov. 8, 2023

(51) Int. Cl. G09G 5/14; G06V 20/62; G06V 30/19; G06V 30/42; G16H 10/60

(52) U.S. Cl. CPC G09G 5/14; G06V 20/62; G06V 30/19; G06V 30/42; G16H 10/60; G09G 2354/00; G09G 2380/08

(57) ABSTRACT

A display data processing device (100) for analysing and/or augmenting display data is provided. The device comprises an input interface (102) configured to receive input display data from an image rendering device (300), and an output interface (104) configured to transmit output display data to at least one display (400) for displaying the output display data at the at least one display (400), wherein the input display data includes user interface data indicative of a graphical user interface (410) displayable at the at least one display. The device further includes a processing circuitry (106) including one or more processors (108), wherein the processing circuitry is configured to determine, based on analysing the input display data, augmentation data for augmenting the input display data, determine, based on analysing the input display data, at least one augmentation position for displaying the augmentation data at the least one display (400), and generate the output display data based on supplementing the input display data with the determined augmentation data and the determined at least one augmentation position.

### FIELD OF THE INVENTION

The present invention generally relates to the analysis and augmentation of display data, which can be displayed at a display to a user. In particular, the present invention relates to a display data processing device for analysing and/or augmenting display data, to a processing system with such processing device, to use of such processing device for augmenting display data, to a method of operating such processing device, to a corresponding computer program, and to a corresponding non-transitory program storage medium storing such a program.

### TECHNICAL BACKGROUND

In many fields of technology and industry data and information can be spread among multiple sources, but should favourably be accessed from or displayed at a single point, such as at a display of a workstation or computer. This is particularly true for the health care or medical sector, where, for example, data or information related to an individual patient can be distributed among various data sources. Examples of data sources in the medical sector can be one or more laboratories, one or more hospitals, one or more medical entities performing one or more medical procedures on the patient, health insurance institutions and many other sources. Similar scenarios can be found in other technical fields, such as for example in process automation or industrial process control.

In order to display data from various sources at a display, for example a display at a computer or workstation, and allowing a user to evaluate the data or corresponding information, the computer is usually interconnected and communicatively coupled with the data sources on a data transfer layer or level. To retrieve data from the various data sources, usually dedicated software is required at the computing device that allows for a data communication and data transfer from the respective data source to the workstation or computing device.

The applicant of the present application, Brainlab AG, has developed and acquired a technology comprising a standalone device that is able to forward, process and augment video signals in real-time. In this context, Brainlab AG acquired the technology developed by the Ayoda GmbH, which had filed the published patent application DE 10 2017 010 351 A1. While the aforementioned technology allows for real-time augmentation by merging or overlaying video signals from multiple sources using said standalone device, a functionality of the device is usually limited to specific use cases, for example for augmenting a video signal from one specific source with another video from another specific source. Also, control for the user is limited.

Therefore, it may desirable to provide for an improved display data processing device and system for analysing and/or augmenting display data that can be displayed at one or more displays.

By way of example, the present invention can be used for medical data processing, for example medical video or image data processing, e.g. in connection with a system such as the one described in detail in DE 10 2017 010 351 A1. The present invention, however, is neither limited to this system nor to the processing of medical data.

Aspects of the present disclosure, examples and exemplary embodiments are disclosed in the following. Different exemplary features of the present disclosure can be combined wherever technically expedient and feasible.

### EXEMPLARY SHORT DESCRIPTION OF THE INVENTION

In the following, a short description of the specific features of the present disclosure is given which shall not be understood to limit the disclosure only to the features or a combination of the features described in this section.

Disclosed herein, is a display data processing device and a data processing system comprising such processing device for analysing and/or augmenting display data. The processing device comprises an input interface configured to receive input display data from an image rendering device. The image rendering device as used herein can, for example, refer to a computing device or computer configured to render display data, also referred to as image data, and output these data to a display for displaying the data or information contained therein.

The display data processing device according to the invention further includes a processing circuitry configured to analyse the input display data, in particular an informational content thereof, and determine augmentation data to supplement the input display data and/or to determine an augmentation position for displaying the augmentation data at the display. The processing device is further configured to supplement the input display data with the augmentation data and the augmentation position, thereby generating output display data, which can then be transmitted via an output interface of the processing device to one or more displays for displaying them to a user. Accordingly, based on analysing the input display data and/or an informational content thereof, the processing device can be configured to determine what augmentation data is to be displayed at which augmentation position at the display.

In an exemplary embodiment, the processing device can be coupled between the image rendering device and the at least one display. Also, the processing device can be configured to analyse the input display data and generate the output display data in real time and/or substantially without latency. In certain embodiments, the processing device according to the present disclosure can be designed or configured as standalone device which can be interconnected with the image rendering device and the at least one computer. Accordingly, the processing device can be designed or configured as physically separate and independent device.

Optionally, the processing device can be communicatively coupled to other devices, systems and/or external data sources. For example, the processing device can be configured to retrieve data from one or more other devices, systems and/or external data sources to augment the input display data with the augmentation data and generate the output display data.

Moreover, in certain embodiments, the processing device can be configured to detect textual and/or numerical information and extract such information from the input display data to determine the augmentation data and/or the augmentation position. This may, for example, involve optical character recognition. In yet further exemplary embodiments, the processing device can be configured analyse a graphical user interface displayed at the display and determine one or both of the augmentation data and the augmentation position based on the analysis. Alternatively or additionally, the processing device can be configured to detect a command input from a user in the input display data and determine the augmentation data and/or the augmentation position based thereon.

Compared to conventional approaches for augmenting display data, the display data processing device according to the invention and its embodiments provide enhanced functionality, versatility, adjustability and operability, for example for the user, as will become apparent to the skilled reader from the present disclosure.

### GENERAL DESCRIPTION OF THE INVENTION

In this section, a summarizing description of the general features of the present invention and disclosure is given for example by referring to possible embodiments.

Aspects of the present disclosure relate to a display data processing device for processing, analysing and/or augmenting display data. Other aspects of the present disclosure relate to a processing system with such processing device, to use of such processing device for augmenting display data, to a method of operating such processing device, to a corresponding computer program, and to a corresponding non-transitory program storage medium storing such a program. It is emphasized that any feature, function, functionality, step, technical effect and/or advantage described hereinabove and hereinbelow with respect to one aspect of the present disclosure, equally applies to any other aspect of the present disclosure.

According to an aspect of the present disclosure, there is provided a display data processing device (also referred to as processing device hereinafter) for processing, analysing and/or augmenting display data. The processing device comprises an input interface configured to receive input display data from an image rendering device, and an output interface configured to transmit output display data to at least one display for displaying the output display data at the at least one display. The input display data includes user interface data indicative of a graphical user interface displayable at the at least one display. The display data processing device further includes a processing circuitry with one or more processors. The processing circuitry is configured to:
determine, based on analysing and/or processing the input display data, augmentation data for augmenting the input display data;
determine, based on analysing and/or processing the input display data, at least one augmentation position for displaying the augmentation data at the least one display, wherein at least one of the augmentation data and the at least one augmentation position is determined based on analysing an informational content of the user interface data; and
generate the output display data based on supplementing and/or augmenting the input display data with the determined augmentation data and the determined augmentation position, such that the output display data is displayable at the least one display and/or such that the augmentation data (or information contained therein) is displayable at the augmentation position at the at least one display.

As will be further elucidated hereinbelow, analysing an informational content of the user interface data contained in the input display data and determining the augmentation data and/or the augmentation position based thereon allows to significantly enhance or improve an overall functionality and versatility of the processing device. In particular, the inventive processing device allows for a computer-implemented augmentation of any sort of input display data with any sort of augmentation data. For example, determining the augmentation data and the augmentation position by analysing the input display data and/or an informational content thereof can allow for an automated detection of what augmentation data or information is to be displayed at which position of the display. Consequential thereto, the processing device can be used to advantage in many different applications for augmenting display data that can be displayed at a display and brought to the attention of a user or operator. For instance, by analysing the input display data with the processing device, feedback from a user or user input can be processed and/or detected, which can allow to control one or more functions of the processing device, another device and/or an external data source coupled thereto, for instance allowing the user to adapt the augmentation to specific needs. Apart from that, the processing device according to the invention can be retrofit to existing image rendering devices, in particular without requiring a modification in hardware or software at the image rendering device. In turn, this can allow for a seamless integration of the processing device at reduced cost and without interfering with or altering a configuration of the image rendering device. These and other technical effects and advantages of the present disclosure and its embodiments will become apparent from the following disclosure.

The input interface of the processing device can refer to or comprise a communication interface or circuitry for communicatively coupling the processing device to the image rendering device and for receiving the input display data. The input interface can be configured for wirelessly coupling the processing device to the image rendering device. Alternatively or additionally, the input interface can be configured for wired communication with the image rendering device. For example, the input interface can include one or more of a display port or interface, a video port or interface, a VGA port or interface, a USB port or interface, an HDMI port or interface, or any other suitable port or interface.

Similarly, the output interface of the processing device can refer to or comprise a communication interface or circuitry for communicatively coupling the processing device to the at least one display and for transmitting the output display data to the at least one display. The output interface can be configured for wirelessly coupling the processing device to the at least one display and/or for wired communication with the image rendering device. For example, the output interface can include one or more of a display port or interface, a video port or interface, a VGA port or interface, a USB port or interface, an HDMI port or interface, or any other suitable port or interface.

It should be noted that the input interface and the output interface can be combined in a communication arrangement or circuitry of the processing device or they can be implemented in the processing device as separate interfaces. Further, it should be noted that the processing device can comprise a plurality of input interfaces for receiving input data from a plurality of image rendering devices. Alternatively or additionally, the processing device can comprise a plurality of output interfaces for transmitting the same or different output display data to a plurality of displays.

As used herein, the image rendering device can refer to a computing device configured to generate display data and output the display data at a display. Accordingly, the image rendering device can be configured to visually display or show the display data at the display, for example in the form of a number of images or frames per unit time. The image rendering device may comprise a graphics processor or any other processor with graphics rendering capability or functionality. Further, the image rendering device may comprise a communication interface communicatively couplable with the input interface of the processing device. Moreover, the image rendering device may be a mobile device or may be fixedly installed. Non-limiting examples of image rendering devices are a general-purpose computer, a handheld, a tablet, a notebook, a smartphone, a server, a special purpose computer or any other image-generating or rendering device configured to display an image or display data at a display. Also, the image rendering device may be designed as standalone device or may be implemented in (or may be part of) another device or system, such as an endoscope, a microscope, a medical device, a medical imaging device, an imaging system, a radiation treatment apparatus, a patient support control, a process control device, or the like.

It should be noted that the image rendering device and the processing device as used herein may refer to physically separate and independent devices. For example, the processing device can comprise a housing surrounding one or more components thereof. Generally, the processing device can be installed near the image rendering device or remote therefrom.

As used herein, the at least one display may refer to or comprise any type of display for visually displaying data or corresponding information contained in the display data. Any reference hereinabove and hereinbelow to “a or the display” includes a plurality of displays. The display may optionally provide further functionality, such as touch control and/or include a speaker to provide acoustic signals to the user, for example. Further, the display may comprise a communication interface communicatively couplable with the processing device for receiving the output display data from the processing device. Also the display may be designed as a standalone device or may be implemented in (or may be part of) another device or system, such as an endoscope, a microscope, a medical device, a medical imaging device, an imaging system, a radiation treatment apparatus, a patient support control, a process control device, or the like.

In the context of the present disclosure, the display data, i.e. the input display data and the output display data, and/or the augmentation data may generally refer to or denote data that can be visually or graphically displayed at the at least one display, for example in the form of one or more images comprising one or more pixels. Accordingly, the input display data, the output display data and/or the augmentation data may refer to or include operational data instructing and/or operationally controlling the display to display the content or information contained in the respective data. Displaying the input display data, the output display data and/or the augmentation data may allow a user to visually perceive an information contained in the respective data. In other words, the input display data, the output display data and/or the augmentation data may comprise and/or be indicative of an information displayable at the at least one display.

An information contained in the input display data and/or the output display data, respectively, may also be referred to herein as informational content of the respective display data. Such informational content can include any graphically or visually displayable and perceptible element or item allowing to convey information to the user or allowing the user to derive information therefrom. Exemplary informational content of the input display data and/or the output display may be or comprise one or more of textual information, numerical information, graphical information, figures, sketches, graphs, one or more displayable objects, colour information or any other information displayable at the display.

As the input display data is supplemented by the augmentation data and the augmentation position, an informational content of the input display data may differ from an informational content of the output display data. In particular, the output display data may comprise at least a part of an informational content of the input display data, for example at least a part of the interface data, and include the augmentation data and the augmentation position in addition thereto.

As used herein, supplementing the input display data with the augmentation data and the augmentation position to generate the output display data may include one or more of incorporating the augmentation data into the input display data, merging the augmentation data with the input display data, combining the input display data with the augmentation data, replacing at least a part of the input display data with the augmentation data, and/or overriding at least a part of the input display data with the augmentation data.

In the context of the present disclosure, the augmentation position may refer to or be indicative of a position or location of the at least one display, at which the augmentation data is to be displayed. Accordingly, the input display data can be supplemented with the augmentation data and the augmentation position, such that the augmentation data can be shown or displayed at the augmentation position at the least one display. By way of example, the augmentation position may be indicative of one or more pixels, such as a range or area of pixels, where the augmentation data or the corresponding information contained therein is to be displayed. Alternatively or additionally, the augmentation position may be indicative of coordinates for displaying the augmentation data at the display.

Therein, the augmentation position may be a position within the graphical user interface indicated by the user interface data and/or may be a position outside of the graphical user interface. Also, a plurality of augmentation data or corresponding information can be displayed at a plurality of different augmentation positions at the display.

As used herein, the graphical user interface can refer to or denote any user interface associated with a software or program running at the image rendering device, which interface is graphically or visually displayable at the display. Therein, the user interface can provide information to the user and/or receive user input from the user to control one or more functions of the image rendering device and/or the program associated with the user interface. A user input from the user may be provided via one or more input devices, such as a keyboard, mouse, touch pad, touch interface, haptic control device, or the like, which can be operatively coupled to the image rendering device. Alternatively or additionally, user input may be received by the image rendering device from the at least one display, for example using a touch control of the display.

According to an embodiment, the processing device is couplable and/or configured for being coupled between the image rendering device and the at least one display. This may comprise connecting the processing device to the image rendering device and the display, for example wirelessly or by wire. For instance, the processing device can be coupled to the image rendering device and the display, such that display data generated or output by the image rendering device is forwarded to the processing device and received as input display data. Accordingly, the processing device can be configured to intercept display data that is usually transmitted from the image rendering device to the display and receive these data as input display data. In this context, the processing device can be considered as augmentation device that augments the input display data with the augmentation data to generate the augmented output display data.

In an example, the processing device can be designed as or implemented in a standalone device which can be coupled via the input interface to the image rendering device and via the output interface to the display. An exemplary hardware implementation of the processing device and its processing circuitry may be an integrated circuitry, such as a field programmable gate array, or any other hardware implementation including one or more processors for data processing.

According to an embodiment, the processing circuitry is configured to analyse the input display data and generate the output display data in real time. Alternatively or additionally, the processing circuitry is configured to analyse the input display data and generate the output display data with a latency that is non-perceptible by a user.

In other words, the augmentation can be performed by the processing device in real-time, for example having a latency in time compared to the input display data of below one frame, e.g. below twenty pixels, below eight pixels, or even below four pixels of a frame.

According to an embodiment, the input display data is rendered by the image rendering device. Alternatively or additionally, the input display data is displayable at the at least one display. By way of example, the input display data and/or the output display data includes one or more image frames or images displayable at the at least one display, for example at a certain number of frames per unit time.

According to an embodiment, the input display data includes textual and/or numerical information, wherein the processing circuitry is configured to determine at least one of the augmentation data and the at least one augmentation position based on extracting the textual and/or numerical information from the input display data. Accordingly, the informational content of the input display data may include one or more of a textual information and numerical information. Therein, at least a part of the input display data may contain or include textual and/or numerical information, which can be displayed at the display. Such textual and/or numerical information may be contained in the user interface data and hence refer to information displayed within the graphical user interface. Alternatively or additionally, however, the textual and/or numerical information can be displayed at the display outside of the graphical user interface, and hence can be contained in a part of input display data other than the user interface data.

In an example, extracting the textual and/or numerical information from the input display data may include detecting and/or identifying the textual and/or numerical information, and optionally separating the textual and/or numerical information from other content of the input display data. Alternatively or additionally, a display position of the textual and/or numerical information, i.e. a position at the display, where the textual and/or numerical information is to be displayed, can be extracted and/or determined by the processing device. Optionally, the augmentation position can be determined based on the determined display position and/or in accordance therewith.

Extracting textual and/or numerical information may generally enable the processing device to determine specifics about the content that is to be display at the display to the user or the content that the user requests to be displayed. In turn, this can enable the processing device to determine augmentation data and/or the augmentation position based on or in accordance with the textual and/or numerical information, thereby providing an augmentation tailored to user specific demands or needs.

According to an embodiment, the processing circuitry is configured to extract and/or determine the textual and/or numerical information from the input display data based on optical character recognition. In other words, the processing device can be configured to apply optical character recognition to at least a part of the input display data, thereby deriving the textual and/or numerical information from the input display data. Applying optical character recognition may generally enable the processing device to further process the extracted information, and for example determine augmentation data related to the extracted textual and/or numerical information, which can then be specifically supplemented by means of the augmentation data.

According to an embodiment, the processing device further comprises a communication circuitry communicatively couplable to an external data source, wherein the processing circuitry is configured to retrieve and/or receive at least a part of the augmentation data from the external data source. The communication circuitry may include one or more communication interfaces for wireless and/or wired communication or connection with the external data source. For example, the communication circuitry may be communicatively coupled to the external data source via one or more of a network interface, a WLAN interface, a Bluetooth connection, a radio frequency interface, an Internet connection, a BUS interface or any other suitable data or communication link. Generally, a communication between the processing device and the external data source can be unidirectional or bi-directional.

Coupling the processing device to an external data source and retrieving at least a part of the augmentation data therefrom can allow to supplement or augment the input display data with data from other sources, without requiring a data connection between the image rendering device and the external source. Accordingly, no modification to the hardware or software of the image rendering device may be required.

In an example, retrieving the augmentation data or at least a part thereof may comprise searching and/or accessing a database stored at the external data source. Alternatively or additionally, retrieving the at least part of the augmentation data from the external data source may comprise operationally controlling, with the processing device, the external data source to transmit the at least part of the augmentation data to the processing device. It is emphasized that a part of the augmentation data or all augmentation data may be retrieved from the external data storage. Also, retrieving the at least part of the augmentation data from the external data source may include retrieving intermediate augmentation data and generating, determining, computing and/or deriving, with the processing device, the augmentation data based on or from the intermediate augmentation data.

According to an embodiment, the augmentation data includes one or more of medical data, video data, medical video data, medical image data, and image data. In particular in the medical field, augmentation using one or more of the aforementioned augmentation data may be advantageous, because various data sources can be combined to provide a comprehensive overview to the user at the display. It is emphasized, though, that the processing device according to the present disclosure can be used to advantage in many other applications, such as for example process automation where sensor data and/or other process-related data could be used as augmentation data and gathered from one or more external data sources.

According to an embodiment, the user interface data includes one or more of at least one information item, at least one state information item and at least one control item of the graphical user interface, wherein the processing circuitry is configured to determine at least one of the augmentation data and the at least one augmentation position based on extracting, from the user interface data, one or more of the at least one information item, the at least one state information item, and the at least one control item of the graphical user interface. Extracting one or more of the at least one information item, the at least one state information item, and the at least one control item of the graphical user interface may include one or more of identifying the corresponding item within the graphical user interface, identifying a position of the corresponding item in the graphical user interface, and/or analysing the corresponding item or information contained therein.

Generally, an information item may refer to or include any visually or graphically displayable item allowing to convey information to the user and/or allowing the user to derive information therefrom. Non-limiting examples are a text box, an item of text, one or more numbers, a string, one or more characters, a symbol, an item in a checklist, a completed item in a checklist, an uncompleted item in a checklist, a pending item in a checklist, an item with predefined colour, a geometrical object, a sketch, a figure, a colour, an object, and the like.

Further, as used herein, a state information item of the graphical user interface may refer to or include a displayable item indicative of a state of the graphical user interface and/or a state of a software or program running at the image rendering device. Non-limiting examples are a menu item of the graphical user interface, a dropdown menu, a tooltip at the graphical user interface, a tab shown in the graphical user interface, and the like.

A control item of the graphical user interface may refer to or include any displayable element or item for controlling one or more functions of the image rendering device or a software running thereon. For instance, a user may control the processing device based on a user input, e.g. via a keyboard, a mouse, a touch pad, or any other user input device coupled to the image rendering device. Non-limiting example for control items are buttons, switches, tabs, menu bars, and the like, which can be shown in the graphical user interface and/or which are actuatable by the user based on a user input.

It should be noted that certain elements or items shown in a graphical user interface may include one or more of an information item, a state information item, and a control item. Accordingly, one or more of such items can be combined in a single element or item displayable at the display.

According to an embodiment, the processing device further comprises a communication circuitry communicatively couplable to an external data source, wherein the processing circuitry is configured to retrieve at least a part of the augmentation data from the external data source based on one or more of the at least one information item, the at least one state information item, and the at least one control item of the graphical user interface extracted from the user interface data.

By way of example, the processing device may analyse the input display data with the user interface date contained therein and identify one or more of an informational item, a state information item and a control item. Further, the processing device may be configured to extract one or more of the identified informational item, the state information item and the control item from the graphical user interface, the input display data and/or the user interface data. The processing device may further be configured to analyse one or more of the extracted informational item, the state information item and the control item and determine one or both the augmentation data and one or more augmentation positions based thereon. For instance, at least a part of the augmentation data may be retrieved from one or more external data sources. Alternatively or additionally, the processing device may generate, compute, and/or derive at least a part of the augmentation data from the determined one or of the extracted informational item, the state information item and the control item.

In a further example, the processing device may be configured to determine one or more augmentation positions based on determining one or more positions of the informational item, the state information item and/or the control item in the graphical user interface. For instance, the processing device may be configured to determine an augmentation position based on a position of one or more of the informational item, the state information item and the control item, such that the augmentation data can be displayed without obscuring the corresponding informational item, the state information item and/or the control item and/or without obscuring any other information, element and/or item displayed at the graphical user interface. Alternatively, the processing device may be configured to determine an augmentation position based on a position of one or more of the informational item, the state information item and the control item, such that the augmentation data displayed at the display can at least partly or entirely overlap with the corresponding informational item, the state information item and/or the control item. Accordingly, the augmentation data can be shown at the display as overlay, which potentially may at least partly obscure, hide and/or override the corresponding informational item, the state information item and/or the control item.

According to an embodiment, the processing circuitry is configured to retrieve the at least part of the augmentation data from the external data source based on comparing one or more data items stored at the external data source with one or more of the at least one information item, the at least one state information item, and the at least one control item of the graphical user interface extracted from the user interface data. Comparing the informational item, the state information item and/or the control item with one or more data items stored at the external data source can allow to link the informational content of the graphical user interface to an informational content of the external data source, thereby allowing to determine the augmentation data tailored to a current demand, need and/or application of the user. Further, this may allow to seamlessly integrate the augmentation data into the input display data, without or with only limited user interaction.

According to an embodiment, the augmentation data and/or the output display data includes query data indicative of a query prompting a user to confirm correctness of one or more of the at least one information item, the at least one state information item, and the at least one control item of the graphical user interface extracted from the user interface data of the input display data. Prompting the user for confirmation may allow to ensure that the correct augmentation data is determined and/or displayed at the display.

Generally, the query indicated by the query data may be displayed at the display as message, icon, overlay, notification and/or any other user-perceptible query, including an acoustic and/or haptic signal, if the display provides such functionality. Accordingly, the query data may refer to operational data for controlling one or more functions of the display.

According to an embodiment, the processing circuitry is further configured to determine a response of the user to the query based on analysing further input display data received subsequent to the input display data. For example, the response of the user may be visually displayable at the display, such that corresponding response data is included by the image rendering device in the further input display data, which can be detected by the processing device. Non-limiting examples of such response can be a mouse gesture, a keyboard input, one or more clicks in the graphical user interface, one or more clicks outside the graphical user interface, a user input at a user input device coupled to the image rendering device, or any combination thereof.

According to an embodiment, the processing circuitry is configured to determine a change of one or more of the at least one information item, the at least one state information item and the at least one control item of the graphical user interface based on comparing the input display data with previous input display data preceding the input display data in time. For example, the processing device may be configured to analyse a stream or sequence of input display data in order to detect and/or determine a user input based on determining the change of one or more of the at least one information item, the at least one state information item and the at least one control item of the graphical user interface. This can, for example, allow to automatically detect what a user is currently requesting to be displayed at the display and determine corresponding augmentation data in response, which can be supplemented with the input display data and displayed at the display as output display data. Alternatively or additionally, the processing device may be configured to detect a response and/or feedback from the user based on the aforementioned comparison with previous display data. Overall, this allows to significantly improve versatility and functionality of the processing device, inter alia, by providing a user-specific augmentation requiring minimum user interaction and by providing operational control of the augmentation to the user. It is to be noted that such user control may be active, i.e. where the user actively controls one or more functions of the processing device, for example actively deciding which augmentation data is to be shown. Alternatively, such user control may be or passive, i.e. where the processing device automatically determines the augmentation data and displays them.

According to an embodiment, the graphical user interface indicated by the user interface data relates to a patient management system and/or contains information about one or more patients, about a medical condition of one or more patients, and/or about a medical treatment of one or more patients. Such information can be contained in one or more informational items, state information items, and/or control items of the graphical user interface. Exemplary patient management systems can be a hospital information system (HIS), a laboratory information system (LIS), an insurance information system or any other information system. Typically, such patient information or management systems store the aforementioned information in one or more databases that can be accessed by the user using the graphical user interface displayed at the display to control a software or program running at the image rendering device.

The patient management system may, for example, be accessed by and/or stored at a nurse PC, an administrative PC in an operation room, an endoscope, a microscope, a medical image-generating device a display showing the operating room schedule information or a digital door sign, a display of an anaesthesia device or any other monitoring device, a Linac- or Couch-Control in a radiotherapy treatment room, a medical physician's office PC, and/or any other image rendering device coupled to a display in a medical institution, such as a lab or hospital.

According to an embodiment, the user interface data includes a patient identification item for uniquely identifying a patient, wherein the processing circuitry is configured to extract the patient identification item from the user interface data to determine at least one of the augmentation data and the at least one augmentation position. For instance, the patient identification item may refer to or include a patient ID, a patient name and/or any other information uniquely associated with the patient. Determining the patient identification item by the processing device may allow to determine and/or compute augmentation data related to the patient, such that an informational content of the input display data can be supplemented with appropriate augmentation data.

According to an embodiment, the processing circuitry is configured to retrieve, via a communication circuitry communicatively couplable to an external data source, at least a part of the augmentation data from the external data source based on the extracted patient identification item. For example, the augmentation data retrieved from the external data source can include medical data associated with the patient. This may allow to provide additional information related to the patient to the user.

According to an embodiment, the augmentation position is a position within the graphical user interface indicated by interface data contained in the input display data. Alternatively or additionally, the processing circuitry is configured to detect the graphical user interface based on analysing the input display data, and to determine the augmentation position based on the detected graphical user interface.

According to an embodiment, the augmentation position is a position within a predefined window or region indicated by the input display data. Alternatively or additionally, the processing circuitry is configured to detect a predefined window or region based on analysing the input display data, and to determine the augmentation position based on the detected predefined window or region.

In an exemplary embodiment, the processing circuitry is configured to detect the predefined window or region based on a colour of at least a part of the predefined window or region. For example, a window or region having a certain colour may be displayed at the display and hence contained in the input display data. The processing device may be configured to analyse the input display data and detect the coloured window or region. Such window or region can, for instance, be provided by a software or program running at the image rendering device. This can include a dedicated software or program as well as a browser application displaying the window or region from a website. Alternatively or additionally, the region can also be contained on a desktop of the image rendering device.

According to an embodiment, the processing circuitry is configured to detect a command input from a user in the input display data, the command input being visually displayable at the at least one display, wherein the processing circuitry is configured to determine at least one of the augmentation data and the augmentation position based on the detected command input. Generally, this may allow to provide operational control of the augmentation to the user, thereby improving an overall functionality and versatility of the processing device.

A command input as used herein may refer to any visually displayable user input, feedback and/or response from the user. A command input may be actively provided by the user or passively. Non-limiting examples of a command input may involve one or more of a mouse gesture, a keyboard input, one or more clicks, or any other command input via a user input device coupled to the image rendering device.

According to an embodiment, the processing circuitry is configured to detect the command input based on identifying one or more of a predefined text input from the user, a predefined numerical input from the user, a predefined cursor movement performed by the user, a predefined click operation performed by the user, a predefined control operation performed by the user at the image rendering device, and a predefined object displayed at the at least one display.

For example, the command input can be a persistent command input, which may be persistently shown at the display, or a transient command input, which may be temporarily shown at the display, wherein the command input can be provided by the user based on controlling the image rendering device and/or one or more functions thereof.

According to an embodiment, the processing circuitry is configured to determine at least one control function associated with the detected command input. The processing circuitry may further be configured to perform the determined at least one control function based on controlling the processing device and/or based on controlling one or more external devices communicatively and/or operatively coupled to the processing device. Accordingly, the processing device may be configured to execute one or more control functions in response to the detected command input, which can include controlling the processing device and/or at least one external data source.

According to an embodiment, the at least one control function includes one or more of recording the input display data, taking a screenshot of the display data, analyse content of the display data, display one or more control elements at the at least one display, retrieving data from one or more external sources, and controlling one or more medical devices couplable to the processing device. Accordingly, additional functionality and control can be provided to the user, which can allow to provide a comprehensive augmentation that can be tailored to the user's needs and/or modified based thereon.

A further aspect of the present disclosure relates to a use of a processing device, as described hereinabove and hereinbelow, and/or a use of a processing system including such processing device for augmenting display data, in particular in the medical field.

According to a further aspect of the present disclosure, there is provided a processing system for analysing and/or augmenting display data, wherein the processing system comprises at least one processing device as described hereinabove and hereinbelow. The processing system may further comprise one or more of an image rendering device for providing input display data to the processing device, at least one display for displaying output display data provided by the processing device, one or more computing devices for providing data or information displayable at the at least one display, and one or more external data sources for providing at least a part of the augmentation data.

It is emphasized that any feature, function, functionality, technical effect and/or advantage described hereinabove and hereinbelow with respect to the processing device, equally applies to the processing system.

According to further aspect, there is provided a method of operating a processing device and/or a processing system as described hereinabove and hereinbelow. The method comprises:
receiving, via an input interface of the processing device, input display data from an image rendering device;
determining, based on analysing the input display data with a processing circuitry of the processing device, augmentation data for augmenting the input display data;
determining, based on analysing the input display data with the processing circuitry of the processing device, at least one augmentation position for displaying the augmentation data at at least one display communicatively couplable with the processing device via an output interface of the processing device; and
generating, with the processing circuitry, output display data based on supplementing the input display data with the determined augmentation data and the determined augmentation position.

It is emphasized that any feature, function, functionality, technical effect and/or advantage described hereinabove and hereinbelow with respect to the processing device or system, can be a feature, function, functionality, step, technical effect and/or advantage of the method, as described hereinabove and hereinbelow.

A further aspect of the present disclosure relates to a computer program, which when executed by a processing device and/or a processing system (and/or a processing circuitry thereof), as described hereinabove and hereinbelow, instructs the processing device or system to perform steps of the method, as described hereinabove and hereinbelow.

A further aspect of the present disclosure relates to a non-transitory computer-readable medium storing a computer program, which when executed by a processing device and/or a processing system (and/or a processing circuitry thereof), as described hereinabove and hereinbelow, instructs the processing device or system to perform steps of the method, as described hereinabove and hereinbelow.

The computer program which, when running on at least one processor (for example, a processor) of the processing device or when loaded into at least one memory thereof, causes the processing device to perform the above-described method.

Such program may alternatively or additionally relate to a (physical, for example electrical, for example technically generated) signal wave, for example a digital signal wave, carrying information which represents the program, for example the aforementioned program, which for example comprises code means which are adapted to perform any or all of the steps of the method.

A computer program stored on a disc is a data file, and when the file is read out and transmitted it becomes a data stream for example in the form of a (physical, for example electrical, for example technically generated) signal. The signal can be implemented as the signal wave which is described herein. For example, the signal, for example the signal wave is constituted to be transmitted via a computer network, for example LAN, WLAN, WAN, for example the internet.

### BRIEF DESCRIPTION OF THE DRAWINGS

In the following, the invention is described with reference to the appended figures which give background explanations and represent specific embodiments of the invention. The scope of the invention is however not limited to the specific features disclosed in the context of the figures, wherein

FIG. 1 illustrates a processing system with a display data processing device according to an illustrative embodiment;

FIG. 2 illustrates a processing system with a display data processing device according to an illustrative embodiment;

FIG. 3 illustrates a processing system with a display data processing device according to an illustrative embodiment; and

FIG. 4 shows a flow chart illustrating steps of a method of operating a display data processing device according to an illustrative embodiment.

The figures are schematic only and not true to scale. Further, like elements shown in the drawings can be referenced by identical or like reference numerals

### DESCRIPTION OF EMBODIMENTS

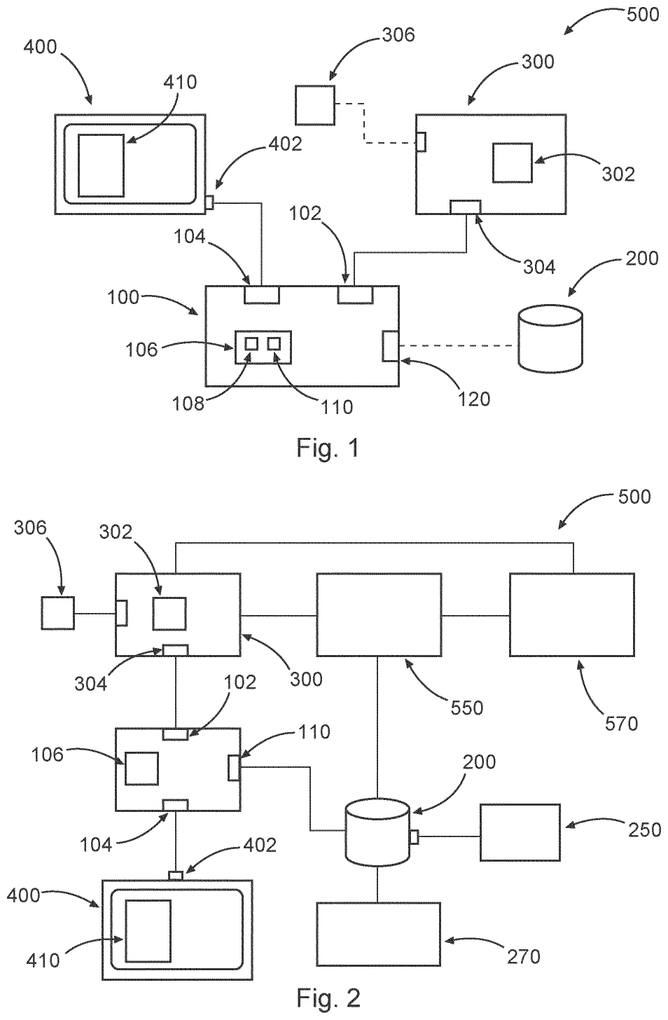

FIG. 1 shows a processing system 500 with a display data processing device 100 according to an illustrative embodiment. It is noted that components of the system 500 other than the processing device 100 are primarily illustrated in FIG. 1 to elucidate the functionality of the processing device 100.

The system 500 comprises an image rendering device 300 comprising one or more processors 302 for data processing and/or rendering one or more images. The image rendering device 300 can generally be configured to render one or more images and output display data that can be displayed at one or more displays 400. Accordingly, the image rendering device 300 can refer to a computing device configured to generate display data and output the display data at a display 400, for example in the form of a number of images or frames per unit time.

The image rendering device 300 further comprises a communication interface 304 communicatively couplable with a corresponding interface 402 of the display 400 and/or couplable with an input interface 102 of the processing device 100, as discussed in more detail hereinabove and hereinbelow. The communication interface 304 of the image rendering device 300 may be configured for wireless or wired data transmission. For instance, the communication interface 304 can include one or more of a display port or interface, a video port or interface, a VGA port or interface, a USB port or interface, an HDMI port or interface, or any other suitable port or interface. The image rendering device 300 may also include a plurality of interfaces for coupling the image rendering device 300 to one or more other devices or systems.

Non-limiting examples of image rendering devices 300 are a general-purpose computer, a handheld, a tablet, a notebook, a smartphone, a server, a special purpose computer or any other image-generating or rendering device configured to display an image or display data at a display. Also, the image rendering device 300 may be designed as standalone device or may be implemented in (or may be part of) another device or system, such as an endoscope, a microscope, a medical device, a medical imaging device, an imaging system, a radiation treatment apparatus, a patient support control, a process control device, or the like. By way of example, the image rendering device 300 may be a nurse PC, an administrative PC in an operation room, an endoscope, a microscope, a medical image-generating device a display showing the operating room schedule information or a digital door sign, a display of an anaesthesia device or any other monitoring device, a Linac- or Couch-Control in a radiotherapy treatment room, a medical physician's office PC, and/or any other image rendering device coupled to a display in a medical institution, such as a lab or hospital.

The image rendering device 300 further includes a user input device 306 operable by the user to provide a user input to the image rendering device 300 and/or to control one or more functions thereof. For example, the user input device 306 can include one or more of a mouse, a keyboard, a touch pad or any other input device.

The system 500 further includes one or more displays 400 configured to display data or information contained therein that is received via a communication interface 402 of the display 400. For instance, the communication interface 402 of the display can include one or more of a display port or interface, a video port or interface, a VGA port or interface, a USB port or interface, an HDMI port or interface, or any other suitable port or interface.

Typically, such display 400 is coupled to the image rendering device 300 to display data provided or rendered by the image rendering device 300 at the display and/or a screen thereof. According to the present disclosure, however, the display data processing device 100 is coupled between the image rendering device 300 and the display 400.

In particular, the processing device 100 comprises an input interface 102 configured to receive input display data from the image rendering device 300 and/or the communication interface 304 thereof. The processing device 100 further comprises an output interface 104 configured to transmit output display data to the at least one display 400 for displaying the output display data at the display 400. Therein, the input display data includes user interface data indicative of a graphical user interface 410 displayable at the display 400.

The input interface 102 and/or the output interface 104 can be configured for wirelessly coupling the processing device 100 to the image rendering device 300 and/or the display 400. Alternatively or additionally, the input interface 102 and/or the output interface 104 can be configured for wired communication with the image rendering device 300 and/or the display 400, respectively. For example, the input interface 102 and/or the output interface 104 can include one or more of a display port or interface, a video port or interface, a VGA port or interface, a USB port or interface, an HDMI port or interface, or any other suitable port or interface.

The processing device 100 further comprises a processing circuitry 106 including one or more processors 108 configured to determine, based on analysing the input display data, augmentation data for augmenting the input display data, and to determine, based on analysing the input display data, at least one augmentation position for displaying the augmentation data at the least one display, wherein at least one of the augmentation data and the at least one augmentation position is determined based on analysing an informational content of the user interface data. The processing circuitry 106 is further configured to generate the output display data based on supplementing the input display data with the determined augmentation data and the determined augmentation position, such that the output display data is displayable at the least one display 400 and/or such that the augmentation data (or information contained therein) is displayable at the determined augmentation position.

The processing circuitry 106 can comprise a data storage 110 or memory 110 for storing data or other information. For example, software instructions instructing the processing device 100 to perform one or more functions, as described hereinabove and as will be described in more detail hereinbelow with reference to subsequent figures, may be stored in the data storage or memory 110.

The processing device 100 further comprises a communication interface 120 for communicatively coupling the processing device 100 to one or more external data sources 200 or devices 200. Any communication standard or protocol can be used for this purpose. The processing device 100 may be configured for wireless or wired connection to the external data source 200. For example, the processing device 100 can be connected to the external data source 200 via a network connection, an Internet connect, a WiFi connection, a Bluetooth connection, a BUS connection or any other connection. The external data source 200 may comprise a database.

FIG. 2 shows a processing system 500 with a display data processing device 100 according to a further illustrative embodiment. Unless stated otherwise, the system 500 of FIG. 2 comprises the same features and components as the system 500 of FIG. 1.

In the exemplary system 500 of FIG. 2 the external data source 200 refers to or includes a server 200, for example a cloud server 200. The server 200 can be operated or controlled directly via an input device 250 coupled thereto. Optionally, the server 200 can be coupled to one or more other devices 270 via a corresponding data connection or link.

The system 500 of FIG. 2 further includes a patient management system 550, for example a hospital network 550. The patient management system 550 can include one or more computing devices and/or one or more databases containing medical data, patient data, patient information, or the like. The patient management system 550 is communicatively coupled with the image rendering device 300 to allows a user to access the patient management system 550, for example by executing corresponding software at the image rendering device 300 or patient management system 550. Information or data provided by the patient management system 550 can be displayed at the display 400, for example in the graphical user interface 410.

The system 500 further comprises a server 570 or other service provider 570 coupled to the image rendering device 300 and allowing the image rendering device 300 to retrieve data and/or information therefrom. Server 570 may for example be a web server 570 that can be accessed by the image rendering device 300 via a browser application executed thereon.

As shown in FIG. 2, the processing device 100 is interconnected between the image rendering device 300 and the display 400. This configuration allows the processing device 100 to analyse and process input display data provided by the image rendering device 300 and supplement these data with the augmentation data and the augmentation position, as described in detail hereinabove and hereinbelow with reference to subsequent figures.

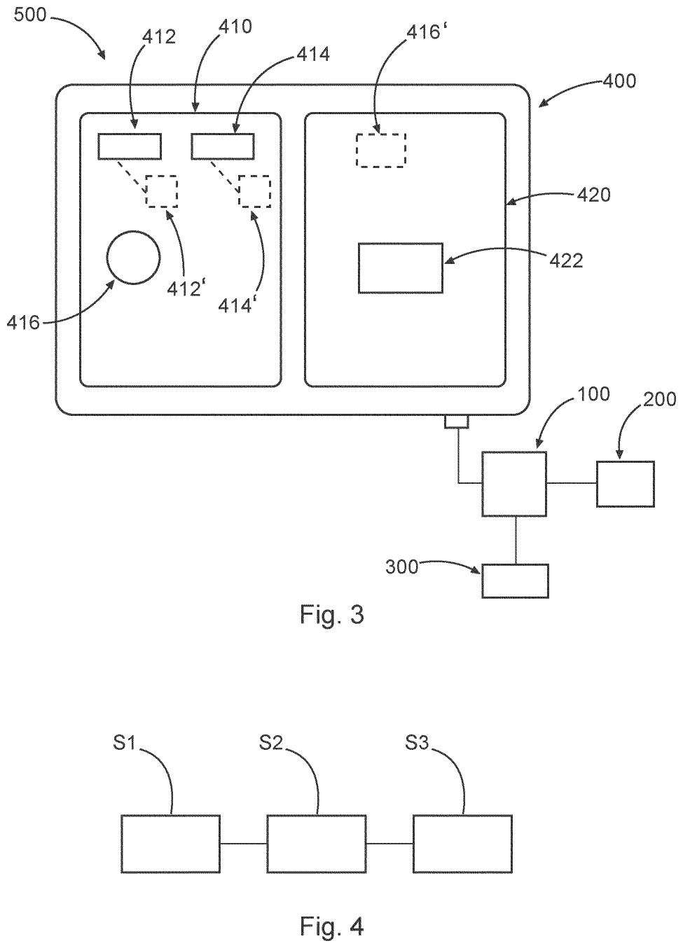

FIG. 3 shows a processing system 500 with a display data processing device 100, a display 400 and an image rendering device 300 according to a further illustrative embodiment. Unless stated otherwise, the system 500 of FIG. 3 comprises the same features and components as the systems 500 of FIGS. 1 and 2.

In the example shown in FIG. 3, the graphical user interface 400 comprises textual and/or numerical information, which is comprised in the user interface data and/or the input display data rendered and/or provided by the image rendering device 300.

The processing device 100 can be configured to analyse the input display data and/or user interface data and extract such numerical and/or textual information from these data in order to determine one or more of the augmentation data and the augmentation position.

In an example, the user interface data includes one or more of at least one information item 412, at least one state information item 414 and at least one control item 416 of the graphical user interface 410, wherein the processing circuitry 100 is configured to determine at least one of the augmentation data and the at least one augmentation position based on extracting, from the user interface data, one or more of the at least one information item 412, the at least one state information item 414, and the at least one control item 416 of the graphical user interface 410.

As described hereinabove, an information item 412 can comprise a text box, an item of text, one or more numbers, a string, one or more characters, a geometrical object, a sketch, a figure, a colour, an object, and the like. A state information item 414 can comprise a menu item of the graphical user interface, a dropdown menu, a tooltip at the graphical user interface, a tab shown in the graphical user interface, and the like. Further, a control item 416 may comprise a button, switch, a tab, a menu bar, or the like.

In an exemplary embodiment, the processing device 100 can be configured to analyse one or more of the information item 412, the state information item 414 and/or the control item 416. This can involve analysing a numerical and/or textual information contained in the corresponding item 412, 414, 416 based on optical character recognition.

Based on such information obtained using optical character recognition, the processing device 100 can for example access the external data source 200 and retrieve augmentation data therefrom, which can then be displayed at the display 400, as indicated by reference numerals 412′ and 414′ in FIG. 3.

Optionally, the processing device 100 may be configured to determine one or more augmentation positions for displaying the augmentation data. This can involve determining a position of the respective item 412, 414, 416 in the graphical user interface 410. As shown in FIG. 3, the augmentation positions determined for the augmentation data 412′, 414′ can be chosen or determined by the processing device 100, such that the augmentation data 412′, 414′ do not obscure the corresponding items 412, 414. Alternatively, however, the augmentation data 412′ 414′ can override or hide the items 412, 414, if desired or appropriate. Alternatively or additionally, an augmentation position outside the graphical user interface 410 may be determined by the processing device 100, for example a position in a predefined region or window 420, and the augmentation data can be displayed there, as indicated by reference numeral 416′ in FIG. 3.

In an exemplary implementation, an information item 412 may include or refer to a patient identification item for uniquely identifying a patient. The processing device 100 can for example extract such information using or applying optical character recognition and use this information to retrieve augmentation data from the external data source 200. Optionally, the processing device 100 can be configured to output a query to the display 400 prompting the user to confirm the information extracted from the patient identification item by the processing device 100.

A response or feedback from the user may, for example, be detected by the processing device 100 by analysing further input display data. For example, such response can be a mouse gesture, a keyboard input, one or more clicks in the graphical user interface 410, one or more clicks outside the graphical user interface 410, a user input at a user input device 306 coupled to the image rendering device 300, or any combination thereof.

In yet a further exemplary implementation, the processing device 100 can be configured to determine a user input at the graphical user interface 410 and compute the augmentation data and/or position in response to the user input. For instance, a user may actuate a menu bar or state information item 414 to switch a state of the graphical user interface 410. This change can be detected by the processing device 100 and augmentation data corresponding to the change initiated by the user can automatically be determined by the processing device 100 and provided at the display 400.

Further exemplary embodiments can include detecting a command input 422 from a user in the input display data, the command input being visually displayable at the at least one display 400. The processing device 100 can for example be configured to detect the command input 422 based on identifying one or more of a predefined text input from the user, a predefined numerical input from the user, a predefined cursor movement performed by the user, a predefined click operation performed by the user, a predefined control operation performed by the user at the image rendering device 300, and a predefined object displayed at the at least one display 400.

In the example shown in FIG. 3, the command input 422 is displayed in a predefined window 420 or region 420 of the display 400. The predefined window or region 420 can be detected by the processing device 100, for example, based on a colour of the window or region 420 or based on any other criteria. The predefined window or region 420 can, for example, refer to a browser window 420 provided by accessing a service provider or server 570 with the image rendering device 300.

Based on the detected command input, the processing device 100 can determine and/or execute one or more control functions associated with the detected command input 422, for example based on controlling the processing device 100 and/or based on controlling one or more external devices 200, 250, 270, 550, 570 communicatively and/or operatively coupled to the processing device 100. A control function can, for instance, include one or more of recording the input display data, taking a screenshot of the display data, analyse content of the display data, display one or more control elements at the at least one display, retrieving data from one or more external data sources 200, and controlling one or more medical devices couplable to the processing device 100.

In the following, various exemplary features, aspects and advantages of the processing device 100 according to the present disclosure are summarized. Generally, the processing device 100 according to the present disclosure allows to overlay augmentation data onto input display data, for example onto a graphical user interface 410, wherein the overlay or augmentation data is only visible at the display 400 and not rendered by the image rendering device 300. This allows to interact with other devices or use other sources by analysing the informational content of the input display data and augmenting same with the augmentation data at the augmentation position. Accordingly, analysis and augmentation can be combined and the augmentation can be based on a result of the analysis.

As discussed above, the image rendering device 300 can be e.g. a computer provided by the hospital running administrative software. The processing device 100 can comprise an input interface 102, such as a video input, and an output interface 104, such as a video output, and optionally another communication interface 120, for example a network connection, to an external data source 200, server 200 or other device 200. The processing device 100 can, for example, be mounted on the back of the display 400 to retrofit the system, and be connected between the image rendering device 300 and the display 400.

The image rendering device 300 renders display data consisting of a user interface 410 and/or comprising corresponding user interface data. Optionally, it can render a predefined region 420, area 420 or window 420, e.g. with a greenscreen or other content. The processing device 100 can receive the input display data and analyse it.

For example, the processing device 100 can extract information, data or content from the input display data, such as patient data, information items 412, state information items 414, e.g. a state of a dropdown menu, and/or control items 416 from the graphical user interface 410. The processing device 100 can also detect state changes, e.g. the scanning of a disposable device, and thus deduce that a surgical event has taken place or will soon take place.

Further, the processing device 100 can strengthen and/or augment the detected information, e.g. by comparing the detected information to other sources 200 with same information. For instance, a detected patient name can be compared with patient names stored at an external data source 200 to gather augmentation data related to the patient from the external source 200.

Also, a training or learning mode of the processing device 100 can be implemented allowing to receive feedback from the user to verify information and “learn”. For example, the processing device 100 could display the detected patient name in the predefined region 420 or window 420 with two buttons below: “OK” and “Incorrect”. Depending on the mouse click in the region 420 or window 420, the patient name detection can be verified or falsified.

Alternatively or additionally, the processing device 100 can detect the difference between the graphical user interface 410 and the region 420 or window 420, for instance based on a colour of the region 420 or window 420.

Alternatively or additionally, the processing device 100 can extract a command or command input 422 from the predefined region 420 or window 420, e.g. a persistent command, such as a label “#video” 422 in the region 420 or window 420, or a transient command input 422, such as a mouse click in the region 420 or window 420, for example that causes one or more pixels, for example a pixel group, to change colour, e.g. from green to black.

Further, the processing device 100 can be configured to display additional output or augmentation data, e.g. partially overwriting the graphical user interface 410. The augmentation can be based on a user input (e.g. a patient name read from the graphical user interface 410), a command input, video data from the external source 200 (e.g. a medical video from another device), a warning message important for the user of the image rendering device 300 but originating from another device or service, and an administrative input not used during everyday clinical use, which means the device is operated “without direct user input”. The administrative input can be configurable, e.g. using an input device 250 connected to server or external data source 200. Video data or image data can preferably be overlaid on the region 420 or window 420.

Generally, a user input can be made available only to the image rendering device 300, for example directly to the graphical user interface 410 and/or at the region 420 or window 420, e.g. in the form of text inputs, drawings, drop-down menu selection, or the like.

Further, the image rendering device 300 could display images or user interface elements 412, 414, 416 that are intended to be augmented by the processing device 100. For example, an endoscope used as image rendering device 300 can display extra menu items providing augmentation data. The processing device 100 can overlay such menu items with custom text or information. If the user selects one of these menu items, the processing device 100 can detect the selection, e.g. by a short blink or other visual cue. Thus, the menu items can be used to select states in applications connected to the processing device 100.

Further, the processing device 100 can acquire augmentation data, e.g. video data via a network or other data connection from one or more external sources 200, e.g. one or more servers 200, for instance based on extracted patient data and/or based on a detected command input 422.

FIG. 4 shows a flow chart illustrating steps of a method of operating a display data processing device 100 and/or a system 500 comprising such device 100 according to an illustrative embodiment. The processing device 100 and/or system 500 can be one of the devices 100 and/or systems 500 described with reference to the foregoing Figures.

In a first step S1, input display data is received via an input interface 102 of the processing device 100 from an image rendering device 300.

In a further step S2, augmentation data for augmenting the input display data is determined based on analysing the input display data with a processing circuitry 106 of the processing device 100. Further, at least one augmentation position for displaying the augmentation data at at least one display 400 communicatively couplable with the processing device 100 via an output interface 104 of the processing device 100 is determined in step S2. Determination of the augmentation data and the at least one augmentation position can be performed sequentially or simultaneously.

In a further step S3, output display data is generated by the processing device 100 based on supplementing the input display data with the determined augmentation data and the determined augmentation position.

Other variations to the disclosed embodiments can be understood and effected by those skilled in the art in practicing the claimed invention, from the study of the drawings, the disclosure, and the appended claims. In the claims the word “comprising” does not exclude other elements or steps and the indefinite article “a” or “an” does not exclude a plurality. A single processor or other unit may fulfill the functions of several items or steps recited in the claims. The mere fact that certain measures are recited in mutually different dependent claims does not indicate that a combination of these measures cannot be used to advantage. A computer program may be stored/distributed on a suitable medium such as an optical storage medium or a solid-state medium supplied together with or as part of other hardware, but may also be distributed in other forms, such as via the Internet or other wired or wireless telecommunication systems. Any reference signs in the claims should not be construed as limiting the scope of the claims.

1. A processing device for analysing and/or augmenting display data, comprising:
an input interface configured to receive input display data from an image rendering device;
an output interface configured to transmit output display data to at least one display for displaying the output display data at the at least one display, wherein the input display data includes user interface data indicative of a graphical user interface displayable at the at least one display; and
one or more processors, configured to:
determine, based on analysing the input display data, augmentation data for augmenting the input display data;
determine, based on analysing the input display data, at least one augmentation position for displaying the augmentation data at the least one display, wherein at least one of the augmentation data and the at least one augmentation position is determined based on analysing an informational content of the user interface data; and
generate the output display data based on supplementing the input display data with the determined augmentation data and the determined at least one augmentation position, such that the output display data is displayable at the least one display.

2. The processing device according to claim 1,
wherein the processing device is couplable and/or configured for being coupled between the image rendering device and the at least one display.

3. The processing device according to claim 1,
wherein the one or more processors is configured to analyse the input display data and generate the output display data in real time; and/or
wherein the one or more processors is configured to analyse the input display data and generate the output display data with a latency non-perceptible by a user.

4. The processing device according to claim 1,
wherein the input display data is rendered by the image rendering device; and/or
wherein the input display data is displayable at the at least one display.

5. The processing device according to claim 1,
wherein the input display data and/or the output display data includes one or more image frames displayable at the at least one display.

6. The processing device according to claim 1,
wherein the input display data includes textual and/or numerical information; and
wherein the one or more processors is configured to determine at least one of the augmentation data and the at least one augmentation position based on extracting the textual and/or numerical information from the input display data.

7. The processing device according to claim 6,
wherein the one or more processors is configured to extract the textual and/or numerical information from the input display data based on optical character recognition.

8. The processing device according to claim 1, further comprising:
a communication circuitry communicatively couplable to an external data source; and
wherein the one or more processors is configured to retrieve at least a part of the augmentation data from the external data source.

9. The processing device according to claim 1,
wherein the augmentation data includes one or more of medical data, video data, medical video data, medical image data, and image data.

10. The processing device according to claim 1,
wherein the user interface data includes one or more of at least one information item, at least one state information item and at least one control item of the graphical user interface; and
wherein the one or more processors is configured to determine at least one of the augmentation data and the at least one augmentation position based on extracting, from the user interface data, one or more of the at least one information item, the at least one state information item, and the at least one control item of the graphical user interface.

11. The processing device according to claim 10, further comprising:
a communication circuitry communicatively couplable to an external data source; and
wherein the one or more processors is configured to retrieve at least a part of the augmentation data from the external data source based on one or more of the at least one information item, the at least one state information item, and the at least one control item of the graphical user interface extracted from the user interface data.

12. The processing device according to claim 11,
wherein the one or more processors is configured to retrieve the at least part of the augmentation data from the external data source based on comparing one or more data items stored at the external data source with one or more of the at least one information item, the at least one state information item, and the at least one control item of the graphical user interface extracted from the user interface data.

13. The processing device according to claim 1,
wherein the augmentation data and/or the output display data includes query data indicative of a query prompting a user to confirm correctness of one or more of the at least one information item, the at least one state information item, and the at least one control item of the graphical user interface extracted from the user interface data of the input display data.

14. The processing device according to claim 13,
wherein the one or more processors is further configured to determine a response of the user to the query based on analysing further input display data received subsequent to the input display data.

15. The processing device according to claim 10,
wherein the one or more processors is configured to determine a change of one or more of the at least one information item, the at least one state information item and the at least one control item of the graphical user interface based on comparing the input display data with previous input display data preceding the input display data in time.

16. The processing device according to claim 1,
wherein the graphical user interface indicated by the user interface data relates to a patient management system and/or contains information about one or more patients, about a medical condition of one or more patients, and/or about a medical treatment of one or more patients.

17. The processing device according to claim 1,
wherein the user interface data includes a patient identification item for uniquely identifying a patient; and
wherein the one or more processors is configured to extract the patient identification item from the user interface data to determine at least one of the augmentation data and the at least one augmentation position.

18. The processing device according to claim 17, further comprising:
a communication circuitry communicatively couplable to an external data source; and
wherein the one or more processors is configured to retrieve at least a part of the augmentation data from the external data source based on the extracted patient identification item.

19. The processing device according to claim 18,
wherein the augmentation data retrieved from the external data source includes medical data associated with the patient.

20. The processing device according to claim 1,
wherein the augmentation position is a position within the graphical user interface indicated by interface data contained in the input display data; and/or
wherein the one or more processors is configured to detect the graphical user interface based on analysing the input display data.

21. The processing device according to claim 1,
wherein the augmentation position is a position within a predefined window or region indicated by the input display data; and/or
wherein the one or more processors is configured to detect a predefined window or region based on analysing the input display data.

22. The processing device according to claim 1,
wherein the one or more processors is configured to detect the predefined window or region based on a colour of at least a part of the predefined window or region.

23. The processing device according to claim 1,
wherein the one or more processors is configured to detect a command input from a user in the input display data, the command input being visually displayable at the at least one display; and
wherein the one or more processors is configured to determine at least one of the augmentation data and the augmentation position based on the detected command input.

24. The processing device according to claim 23,
wherein the one or more processors is configured to detect the command input based on identifying one or more of a predefined text input from the user, a predefined numerical input from the user, a predefined cursor movement performed by the user, a predefined click operation performed by the user, a predefined control operation performed by the user at the image rendering device, and a predefined object displayed at the at least one display.

25. The processing device according to claim 23,
wherein the command input is a persistent command input or a transient command input provided by the user based on controlling the image rendering device.

26. The processing device according to claim 23,
wherein the one or more processors is configured to determine at least one control function associated with the detected command input.

27. The processing device according to claim 26,
wherein one or more processors is further configured to perform the determined at least one control function based on controlling the processing device and/or based on controlling one or more external devices communicatively and/or operatively coupled to the processing device.

28. The processing device according to claim 26,
wherein the at least one control function includes one or more of recording the input display data, taking a screenshot of the display data, analyse content of the display data, display one or more control elements at the at least one display, retrieving data from one or more external sources, and controlling one or more medical devices couplable to the processing device.

29. (canceled)

30. A system for analysing and/or augmenting display data, comprising:
a processing device having at least one processor executing instructions causing the at least one processor to:
determine, based on analysing the input display data, augmentation data for augmenting the input display data;
determine, based on analysing the input display data, at least one augmentation position for displaying the augmentation data at the least one display, wherein at least one of the augmentation data and the at least one augmentation position is determined based on analysing an informational content of the user interface data; and
generate the output display data based on supplementing the input display data with the determined augmentation data and the determined at least one augmentation position, such that the output display data is displayable at the least one display;
an image rendering device for providing input display data to the processing device;
at least one display for displaying output display data provided by the processing device;
one or more computing devices for providing data or information displayable at the at least one display; and
one or more external data sources for providing at least a part of the augmentation data.

31. A computer implemented method comprising:
receiving, via an input interface, input display data from an image rendering device;
determining, based on analysing the input display data, augmentation data for augmenting the input display data;
determining, based on analysing the input display data, at least one augmentation position for displaying the augmentation data at the at least one display; and
generating, output display data based on supplementing the input display data with the determined augmentation data and the determined augmentation position.

32. (canceled)

33. A non-transitory computer-readable medium storing instructions which, when executed by at least one processor, causes the at least processor to:
determine, based on analysing input display data, augmentation data for augmenting the input display data;
determine, based on analysing the input display data, at least one augmentation position for displaying the augmentation data on at least one display, wherein the augmentation data and the augmentation position are determined based on analysing an informational content of the user interface data; and
generate output display data based on supplementing the input display data with the determined augmentation data and the determined at least one augmentation position, such that the output display data is displayable on the at least one display.
